(19) United States
(12) Patent Application Publication
(10) Pub. No.: US 2002/0193883 A1
(43) Pub. Date: Dec. 19, 2002

(54) Injectable porous bone graft materials

(76) Inventor: John F. Wironen, Alachua, FL (US)

Correspondence Address: VAN DYKE & ASSOCIATES, P.A., 1630 HILLCREST STREET, ORLANDO, FL, 32803, US

(21) Appl. No.: 10/056,217

(22) Filed: Jan. 25, 2002

Related U.S. Application Data
(60) Provisional application No. 60/263,972, filed on Jan. 25, 2001.

(51) Int. Cl. A61F 2/28

(52) U.S. Cl. 623/23.56

(57) ABSTRACT

A bone-like implant capable of increasing its porosity in situ comprising at least one bone-like compound with at least one hydrophobic carrier, or a degradable component. The bone-like implant includes its manufacture and methods of use. One aspect of the bone-like implant is to provide a method of repairing a bone defect or related injuries. The bone-like implant includes several embodiments capable of increasing its porosity in situ.

### CROSS-REFERENCE TO RELATED APPLICATIONS

This application is filed as a non-provisional claiming right of priority date of application Serial No. 60/263,972, filed on Jan. 25, 2001, under 35 U.S.C. §119(e).

### FIELD OF THE INVENTION

This invention relates to a new bone-like implant, more specifically, a bone-like implant capable of increasing its porosity in situ comprising at least one bone-like compound with at least one hydrophobic carrier, or a degradable component.

### BACKGROUND OF THE INVENTION

Much progress has been made in the field of bone pastes and cements in recent years. For example, REGENAFIL produced by Regeneration Technologies, Inc is an injectable bone graft paste that has been shown to have superior osteoinductive properties without the adverse side effects and toxicities displayed by other products. “An Unexpected Outcome During Testing of Commercially Available Demineralized Bone Graft Materials,” North American Spine Society Proceedings, 15^{th} Annual Meeting, (October 2000). The REGENAFIL product comprises precious, allograft demineralized bone materials as one of its components. Ultimately, these precious materials have a finite supply and, consequently, can be expensive. Depending on the application, it is not always necessary to utilize products containing allograft bone materials to repair bone defects. A number of bone graft substitutes have been developed for use in orthopedic applications, but these substitutes tend to possess undesired drawbacks, such as, for example, low porosity, or not being injectable or moldable. Accordingly, there is a need in the art for bone graft substitutes having increased porosity and which can be injected and easily administered to the site of need.

### SUMMARY OF THE INVENTION

The present invention is directed to a new bone-like implant including its manufacture and methods of use. The bone-like implants are capable of increasing its porosity in situ comprising at least one bone-like compound with at least one hydrophobic carrier, or a degradable component. One aspect of the bone-like implant is to provide a method of repairing a bone defect or related injuries. Accordingly, there are several bone-like implants capable of increasing its porosity in situ. The first embodiment of the bone-like implant comprises at least one bone-like compound mixed with a hydrophobic carrier and is further combined with an aqueous phase or component. The second embodiment is a method of mixing the bone-like implant comprises at least one bone-like compound and hydrophobic carrier whereby carrier is in a syringe-like container and added to the dry bone-like compound to form a dry ingredient mixture which is then taken up into the syringe for administration at a desired site for implantation. Another embodiment of the bone implant comprises at least one bone-like compound mixed with a degradable component which can include gas-producing degradable compounds and an effective amount of an acid.

Accordingly, it is one object of this invention to provide a method of repairing a bone defect and injury.

A further object of this invention is to provide a bone-like implant leaving a porous bone-like implant at the site of need.

Still another object of this invention is to provide a method of making an injectable bone graft material that has porosity to aid in osteoconduction.

Yet another object of this invention is to provide a bone-like implant capable of increasing its porosity in situ.

The foregoing has outlined some of the more pertinent objectives of the present invention. These objectives should be construed to be merely illustrative of some of the more prominent features and applications of the invention. Applying the disclosed invention in a different manner by modifying the invention will be described and can attain many other beneficial results.

It is to be understood that the foregoing general description and the following detailed description are exemplary and explanatory only and are not to be viewed as being restrictive of the present, as claimed. These and other objects, features and advantages of the present invention will become apparent after a review of the following detailed description of the disclosed embodiments and the appended claims.

### DESCRIPTION OF THE PREFERRED EMBODIMENTS

One aspect of the subject invention pertains to a method of making an injectable bone graft material that has porosity to aid in osteoconduction. According to a specific embodiment, bone-like minerals requiring aqueous sintering are mixed in a hydrophobic carrier. Examples of such types of materials include tri-, di-, or mono-calcium phosphate, potassium phosphates, calcium sulphates, hydroxyapatites, or bioactive glasses such as BIOGLASS®. All of the following embodiments including bone-like minerals or compound can comprise of an osteogenic, vasogenic, neorogenic, or like growth factors, hormone, or protein. At least one osteogenic factor or protein comprises one or more selected from the group consisting of platelet derived growth factors (PDGF), transforming growth factors (TGF-.beta.), insulin-like growth factors (IGF's), fibroblast growth factors (FGF's), epidermal growth factor (EGF), human endothelial cell growth factor (ECGF), granulocyte macrophage colony stimulating factor (GM-CSF), nerve growth factor (NGF), vascular endothelial growth factor (VEGF), cartilage derived morphogenetic protein (CDMP), and bone morphogenetic proteins (BMP's). In addition, one or more osteogenic protein can include OP-1, OP-2, BMP2, BMP3, BMP4, BMP9, DPP, Vg-1, 60A, and Vgr-1, including naturally sourced and recombinant derivatives of the foregoing. Another preferred embodiment of the present invention includes the subject bone-like implant further comprises demineralized bone matrix, preferably in particulate or powder form.

Preferably, hydrophobic carriers suitable with this aspect of the subject invention are physiologically acceptable and have minimal deleterious side effects such as toxicity or antigenicity. Examples of such carriers include squalene, hydrophobic proteins, lipids, amphophyllic proteins or glycoproteins; wax-like low molecular weight biodegradable polymers like low molecular weight polyglycolic acid, a copolymer of polycaprolactone and polyglycolic acid, or other polyesters, polyanhydrides, polyamines, nylons etc.; or combinations of the foregoing. Before administration of the subject materials, the mineral/carrier mixture is combined with an aqueous phase (e.g., water, saline, blood, etc.) and upon injection, the combined mixture sets up in situ as a heterogeneous mixture. Subsequently, the hydrophobic carrier dissolves or degrades away, in vivo, thereby leaving a sintered or curing bone-like mineral material having interconnected porosity.

Bone-like minerals may be provided as powders, which may be premixed or may be provided as separate components to be mixed in the carrier. The carrier may be provided in a separate container, conveniently a syringe, where the syringe may be used to add the carrier to the dry components, the dry ingredients mixed and then taken up into the syringe for administration at the desired site. U.S. patent application Ser. No. 09/474,276 provides a preferred method of reconstituting paste materials with a fluid that could be adapted to mixing the dry components with the hydrophobic carrier. Those skilled in the art will appreciate in view of the teachings herein that other conventional means of administration, such as through a catheter or manual packing, would be suitable for delivery of the subject materials.

The disclosures of U.S. Pat. Nos. 5,954,867, RE 33,161, and 5,997,624 are expressly incorporated herein by reference to the extent that they are not inconsistent with the teachings herein. These references teach various calcium phosphate compositions that could be adapted for use with the subject methods for producing an injectable bone-like graft material that becomes porous in situ.

In another embodiment, bone-like minerals are mixed with a degradable agent. Prior to administration, the mixture is hydrated such that the mixture remains injectable but sets up as two components: mineral component and degradable component. When the rapidly degradable component degrades, a porous implant remains at the site of administration. Degradable agents suitable for use with the subject invention include gelatin; polyglycolic acid and other polyhydroxypolyesters; cross-linked albumin; collagen; other proteins, polysaccharides, glycoproteins; or combinations of the foregoing.

According to another embodiment, porous injectable graft materials are optionally made by adding a degradable gas-producing compound. As gas bubbles are produced from the gas-producing compound, pores are formed in the bone-like materials. The size of the pores are preferably controlled by adjusting the amount of gas-producing compound and the viscosity of the mineral matrix in the fluid used to mix the materials. In a specific embodiment, sodium bicarbonate and/or calcium bicarbonate is added to a bone-like mineral powder and a precise amount of acid (e.g. citric acid, formic, acetic, phosphoric acids, HCL) is added to the mixing fluid. The acidity of the mixing fluid causes carbon dioxide to be released from the sodium bicarbonate, wherein the carbon dioxide ultimately forms pores in the bone-like materials. In an alternative embodiment, hydrogen peroxide is combined with peroxidase in the graft material. The peroxidase releases oxygen from the hydrogen peroxide which has the added advantage of sterilizing the wound site.

What is claimed is:

1. An injectable bone-like implant capable of increasing its porosity in situ comprising at least one bone-like compound and a hydrophobic carrier.

2. The injectable bone-like implant according to claim 1, wherein said bone-like compound is capable of aqueous sintering or curing.

3. The injectable bone-like implant according to claim 1, wherein said at least one bone-like compound is tricalcium phosphate, dicalcium phosphate, or monocalcium phosphate, potassium phosphate, calcium sulphate, hydroxyapatite, bioactive glass or combinations thereof.

4. The injectable bone-like implant according to claim 1, wherein said bone-like implant further comprises at least one of osteogenic, vasogenic, neorogenic, or like growth factors, hormone, or protein.

5. The injectable bone-like implant according to claim 4, wherein said at least one osteogenic factor or protein is selected from the group consisting of platelet derived growth factors (PDGF), transforming growth factors (TGF-.beta.), insulin-like growth factors (IGF's), fibroblast growth factors (FGF's), epidermal growth factor (EGF), human endothelial cell growth factor (ECGF), granulocyte macrophage colony stimulating factor (GM-CSF), nerve growth factor (NGF), vascular endothelial growth factor (VEGF), cartilage derived morphogenetic protein (CDMP), bone morphogenetic proteins (BMP's), and combinations of the foregoing

6. The injectable bone-like implant according to claim 4, wherein one or more said osteogenic protein is selected from the group consisting of OP-1, OP-2, BMP2, BMP3, BMP4, BMP9, DPP, Vg-1, 60A, and Vgr-1, including naturally sourced and recombinant derivatives of the foregoing.

7. The injectable bone-like implant according to claim 1, wherein said bone-like implant further comprises demineralized bone matrix.

8. The injectable bone-like implant according to claim 1, wherein said hydrophobic carrier is squalene, hydrophobic proteins, lipids, amphophyllic proteins, glycoproteins, polyesters, polyanhydrides, polyamines, nylons, or combinations thereof.

9. The injectable bone-like implant according to claim 1, wherein said hydrophobic carrier comprises a wax-like low molecular weight biodegradable polymers selected from the group consisting of polyglycolic acid, a copolymer of polycaprolactone and polyglycolic acid, or other polyesters, polyanhydrides, polyamines, nylons, or any combinations thereof.

10. The injectable bone-like implant according to claim 1, further comprising an aqueous component.

11. The injectable bone-like implant according to claim 10, wherein said aqueous component is water, saline, blood, or the like, or any combination thereof.

12. A method of producing an injectable bone-like implant, wherein said implant is capable of increasing its porosity in situ, said method comprising the steps of:
mixing at least one bone-like compound in a hydrophobic carrier; and
concurrently or subsequent to said mixing step, combining said at least one bone-like compound and said hydrophobic carrier with an aqueous phase to form a combined mixture.

13. The method according to claim 12, wherein said at least one bone-like compound is tricalcium phosphate, dicalcium phosphate, or monocalcium phosphate, potassium phosphate, calcium sulphate, hydroxyapatite, bioactive glass or combinations thereof.

14. The method according to claim 12, wherein said bone-like implant further comprises at least one of osteogenic, vasogenic, neorogenic, or like growth factors, hormone, or protein.

15. The method according to claim 14, wherein said at least one osteogenic factor or protein is selected from the group consisting of platelet derived growth factors (PDGF), transforming growth factors (TGF-.beta.), insulin-like growth factors (IGF's), fibroblast growth factors (FGF's), epidermal growth factor (EGF), human endothelial cell growth factor (ECGF), granulocyte macrophage colony stimulating factor (GM-CSF), nerve growth factor (NGF), vascular endothelial growth factor (VEGF), cartilage derived morphogenetic protein (CDMP), bone morphogenetic proteins (BMP's), and combinations of the foregoing.

16. The method according to claim 14, wherein one or more said osteogenic protein is selected from the group consisting of OP-1, OP-2, BMP2, BMP3, BMP4, BMP9, DPP, Vg-1, 60A, and Vgr-1, including naturally sourced and recombinant derivatives of the foregoing.

17. The method according to claim 12, wherein said method comprises adding demineralized bone matrix to said bone-like compound.

18. The method according to claim 12, wherein said hydrophobic carrier is squalene, hydrophobic proteins, lipids, amphophyllic proteins, glycoproteins, polyesters, polyanhydrides, polyamines, nylons, or combinations thereof.

19. The method according to claim 12, wherein said hydrophobic carrier comprises a wax-like low molecular weight biodegradable polymers selected from the group consisting of polyglycolic acid, a copolymer of polycprolactone and polyglycolic acid, or other polyesters, polyanhydrides, polyamines, nylons, or any combinations thereof.

20. The method according to claim 12, further comprises an aqueous component.

21. The method according to claim 20, wherein said aqueous component is water, saline, blood, or the like, or any combination thereof.

22. The method according to claim 12, wherein said step of mixing at least one bone-like compound in a hydrophobic carrier further comprises the step of:
providing said at least one bone-like compound in a dried powdered form, and reconstituting said dried bone-like compound with said hydrophobic carrier.

23. A method of repairing a bone defect and injury comprising the steps of:
mixing at least one bone-like compound in a hydrophobic carrier;
concurrently or subsequent to said mixing step, combining said at least one bone-like compound and said hydrophobic carrier with an aqueous phase to form a combined mixture; and
administering an amount of said combined mixture in a patient at a site of need; wherein said combined mixture sets up in situ, thereby leaving a porous bone-like implant at the site of need.

24. An injectable bone-like implant capable of increasing its porosity in situ comprising at least one bone-like compound and at least one degradable component.

25. The injectable bone-like implant according to claim 24, wherein said at least one bone-like compound is tricalcium phosphate, dicalcium phosphate, or monocalcium phosphate, potassium phosphate, calcium sulphate, hydroxyapatite, bioactive glass or combinations thereof.

26. The injectable bone-like implant according to claim 24, wherein said bone-like implant further comprises at least one of osteogenic, vasogenic, neorogenic, or like growth factors, hormone, or protein.

27. The injectable bone-like implant according to claim 26, wherein said at least one osteogenic factor or protein is selected from the group consisting of platelet derived growth factors (PDGF), transforming growth factors (TGF-.beta.), insulin-like growth factors (IGF's), fibroblast growth factors (FGF's), epidermal growth factor (EGF), human endothelial cell growth factor (ECGF), granulocyte macrophage colony stimulating factor (GM-CSF), nerve growth factor (NGF), vascular endothelial growth factor (VEGF), cartilage derived morphogenetic protein (CDMP), bone morphogenetic proteins (BMP's), and combinations of the foregoing.

28. The injectable bone-like implant according to claim 26, wherein one or more said osteogenic protein is selected from the group consisting of OP-1, OP-2, BMP2, BMP3, BMP4, BMP9, DPP, Vg-1, 60A, and Vgr-1, including naturally sourced and recombinant derivatives of the foregoing.

29. The injectable bone-like implant according to claim 24, wherein said bone-like implant further comprises demineralized bone matrix.

30. The injectable bone-like implant according to claim 24, wherein said at least one degradable component is gelatin, polyglycolic acid and other polyhydroxypolyesters, cross-linked albumin, collagen, proteins, polysaccharides, glycoproteins, or any combination thereof.

31. The injectable bone-like implant according to claim 24, wherein said at least one degradable component a degradable gas-producing compound and an effective amount of an acid.

32. The injectable bone-like implant according to claim 31, wherein said degradable gas-producing compound is sodium bicarbonate, calcium bicarbonate, or the like, or any combination thereof.

33. The injectable bone-like implant according to claim 31, wherein said acid is citric acid, formic acid, acetic phosphoric acids, or HCl.

34. The injectable bone like implant according to claim 31, wherein said degradable gas-producing component is hydrogen peroxide and peroxidase.

35. A method of producing an injectable bone-like implant, wherein said implant is capable of increasing its porosity in situ, said method comprising the steps of:
mixing at least one bone-like compound in a degradable component; and
concurrently or subsequent to said mixing step, combining said at least one bone-like compound and said degradable component with an aqueous phase to form a combined mixture.

36. The method according to claim 35, wherein said at least one bone-like compound is tricalcium phosphate, dicalcium phosphate, or monocalcium phosphate, potassium phosphate, calcium sulphate, hydroxyapatite, bioactive glass or combinations thereof.

37. The method according to claim 35 wherein said bone-like implant further comprises at least one of osteogenic, vasogenic, neorogenic, or like growth factors, hormone, or protein.

38. The method according to claim 37, wherein said at least one osteogenic factor or protein is selected from the group consisting of platelet derived growth factors (PDGF), transforming growth factors (TGF-.beta.), insulin-like growth factors (IGF's), fibroblast growth factors (FGF's), epidermal growth factor (EGF), human endothelial cell growth factor (ECGF), granulocyte macrophage colony stimulating factor (GM-CSF), nerve growth factor (NGF), vascular endothelial growth factor (VEGF), cartilage derived morphogenetic protein (CDMP), bone morphogenetic proteins (BMP's), and combinations of the foregoing.

39. The method according to claim 37, wherein one or more said osteogenic protein is selected from the group consisting of OP-1, OP-2, BMP2, BMP3, BMP4, BMP9, DPP, Vg-1, 60A, and Vgr-1, including naturally sourced and recombinant derivatives of the foregoing.

40. The method according to claim 35, wherein said method comprises adding demineralized bone matrix to said bone-like compound.

41. The method according to claim 35, wherein said at least one degradable component is gelatin, polyglycolic acid and other polyhydroxypolyesters, cross-linked albumin, collagen, proteins, polysaccharides, glycoproteins, or any combination thereof.

42. The method according to claim 35, further comprising an aqueous component.

43. The method according to claim 42, wherein said aqueous component is water, saline, blood, or the like, or any combination thereof.

44. The method according to claim 35, wherein said at least one degradable component comprises a degradable gas-producing compound and an effective amount of an acid.

45. The method according to claim 44, wherein said degradable gas-producing compound is sodium bicarbonate, calcium bicarbonate, or the like, or any combination thereof.

46. The method according to claim 44, wherein said acid is citric acid, formic acid, acetic phosphoric acids, or HCl.

47. The method according to claim 44, wherein said degradable gas-producing component is hydrogen peroxide and peroxidase.

48. A method of repairing a bone defect and injury comprising the steps of:
mixing at least one bone-like compound with at least one degradable component;
combining said at least one bone-like compound and at least one degradable substance with an aqueous phase to form a combined mixture; and
administering an amount of said combined mixture in a patient at a site of need;
wherein said combined mixture sets up in situ, thereby leaving a porous bone-like implant at the site of need.

49. The method according to claim 48, wherein said at least one bone-like compound is tricalcium phosphate, dicalcium phosphate, or monocalcium phosphate, potassium phosphate, calcium sulphate, hydroxyapatite, bioactive glass or combinations thereof.

50. The method according to claim 48, wherein said bone-like implant further comprises at least one of osteogenic, vasogenic, neorogenic, or like growth factors, hormone, or protein.

51. The method according to claim 50, wherein said at least one osteogenic factor or protein is selected from the group consisting of platelet derived growth factors (PDGF), transforming growth factors (TGF-.beta.), insulin-like growth factors (IGF's), fibroblast growth factors (FGF's), epidermal growth factor (EGF), human endothelial cell growth factor (ECGF), granulocyte macrophage colony stimulating factor (GM-CSF), nerve growth factor (NGF), vascular endothelial growth factor (VEGF), cartilage derived morphogenetic protein (CDMP), bone morphogenetic proteins (BMP's), and combinations of the foregoing.

52. The method according to claim 50, wherein one or more said osteogenic protein is selected from the group consisting of OP-1, OP-2, BMP2, BMP3, BMP4, BMP9, DPP, Vg-1, 60A, and Vgr-1, including naturally sourced and recombinant derivatives of the foregoing.

53. The method according to claim 48, wherein said method comprises adding demineralized bone matrix to said bone-like compound.

54. The method according to claim 48, wherein said aqueous component is water, saline, blood, or the like, or any combination thereof.

55. The method according to claim 48, wherein said at least one degradable component comprises a degradable gas-producing compound and an effective amount of an acid.

56. The method according to claim 55, wherein said degradable gas-producing compound is sodium bicarbonate, calcium bicarbonate, or the like, or any combination thereof.

57. The method according to claim 55, wherein said acid is citric acid, formic acid, acetic phosphoric acids, or HCl.

58. The method according to claims 55, wherein said degradable gas-producing component is hydrogen peroxide and peroxidase.
